(19) United States
(12) Patent Application Publication
(10) Pub. No.: US 2023/0099235 A1
(43) Pub. Date: Mar. 30, 2023

(54) NUTRITION TRACKING SYSTEM THAT UTILIZES NUTRITION INFORMATION ON PACKAGED FOOD LABEL AND GENERATES GRAPH INDICATIVE OF HEALTH RATING

(71) Applicant: Smile Food System, LLC, Ann Arbor, MI (US)

(72) Inventor: Jack Fishstrom, Ann Arbor, MI (US)

(21) Appl. No.: 17/820,569

(22) Filed: Aug. 18, 2022

Related U.S. Application Data
(60) Provisional application No. 63/261,844, filed on Sep. 30, 2021.

(51) Int. Cl. G16H 20/60; G09B 19/00

(52) U.S. Cl. CPC G16H 20/60; G09B 19/0092

(57) ABSTRACT

A method for tracking nutrition includes obtaining, by a user device, data representative of at least a portion of a nutrition facts table of a food item. The method also includes determining, by a processor of the user device, using the data, a plurality of nutrition values. Each nutrition value represents an amount of a respective nutrient present in the food item. The method includes assigning, by the processor, a nutrition weight to each respective nutrient and plotting, by the processor, the nutrition values on a graph based on the nutrition weights of each respective nutrient. The plot forms a pattern indicative of a healthiness level of the food item. The method also includes displaying the plot on a display of the user device.

### CROSS REFERENCE TO RELATED APPLICATION

The present application claims the filing benefits of U.S. provisional application Ser. No. 63/261,844, filed Sep. 30, 2021, which is hereby incorporated herein by reference in its entirety.

### FIELD OF THE INVENTION

The present invention relates generally to determining nutrition information and generating graphics indicative of a health rating.

### BACKGROUND OF THE INVENTION

Nutrition information generally must be provided with the sale of food. The nutrition information is typically provided via a food label that is often difficult to quickly parse and understand for the average consumer.

### SUMMARY OF THE INVENTION

A method for tracking nutrition includes obtaining, by a user device, data representative of at least a portion of a nutrition facts table of a food item. The method includes determining, by a processor of the user device, and via processing the data, a plurality of nutrition values. Each nutrition value represents an amount of a respective nutrient present in the food item (particularly fiber, sugar, and protein). The method includes assigning, by the processor, a nutrition weight to each respective nutrient. The method includes plotting, by the processor, the nutrition values on a graph based on the nutrition weights of each respective nutrient, the plot forming a pattern indicative of a healthiness or a healthiness rating of the food item. The method includes displaying the plot on a display of the user device.

These and other objects, advantages, purposes and features of the present invention will become apparent upon review of the following specification in conjunction with the drawings.

### BRIEF DESCRIPTION OF THE DRAWINGS

FIG. 1 is an image of a conventional food label;

FIGS. 2-6 are exemplary plots of a portion of nutritional information determined from a nutritional label; and

FIG. 7 is a schematic view of an example computing device that may be used to implement the systems and methods described herein.

### DESCRIPTION OF THE PREFERRED EMBODIMENTS

Nutrition labels are found on most food items sold to consumers. The most common label is a conventional nutrition facts table that lists a large quantity of nutrition information using multiple rows and/or columns. The dense block of information is often difficult to parse quickly (e.g., as a customer reviews the label while shopping at a store) to determine the nutritional qualities of the food item.

Different nutrients illustrated on nutrition facts tables have different influences on diet. For example, fiber slows digestion and lowers a rate at which rate sugar is absorbed, which helps reduce or eliminate spikes in blood glucose. Without spikes, a person experiences a more smooth hunger without cyclical ups and downs of hunger, fast eating, hunger, more eating, etc. Overall, fiber helps the body fully absorb calories present within the food along with other nutritional benefits of the foods you eat during the day, without cravings for food brought on by “sugar crashes.” With a high-fiber diet, a person may feel full after eating. Fiber also helps in digestion, keeping your bowel movements regular and your intestines clean. Fiber may reduce or preclude constipation and may lower risk of colon cancer. A cracker or piece of bread, for example, with higher fiber than a competing item is generally considered the better choice.

According to many health experts from various specialty areas (such as cardiology, neurology, immunology, etc.), the human body does not need added sugar in foods. These added sugars can be simple sugars (honey, sugar, etc.) or highly processed, such as high-fructose corn syrup. Of these, the simple forms (i.e., fructose, glucose, and lactose) are generally considered better (i.e., less detrimental), but are still unnecessary for good health. Sufficient sugars are found naturally in many healthful foods that are typically consumed, such as fruits, vegetables, and dairy products. Sugars are a type of carbohydrate and provide calories, which are needed on a daily basis for energy for the brain and other organs and muscles. Thus, sugar has a positive function, but a person can obtain a sufficient amount from natural and whole foods without added sugars. Avoiding added sugar, however, is nearly impossible, as food manufacturers put sugar in nearly everything, including items not generally associated with sugar and sweets, such as spaghetti sauce and salad dressing. Thus, some health experts recommend people attempt to limit sugar intake as much as possible, allowing for an occasional dessert or treat.

Excessive sugar may have severe ill health effects. Heart disease, obesity, diabetes, and depression are thought to be manifested by over-consumption of sugar. Overall, added sugar may be of very little value to the body and provides very little essential nutritional benefit, especially considering that it naturally occurs in foods that are high in fiber and protein as well. Aside from helping foods to taste sweet, added sugar is generally not beneficial to a person's health. Moreover, eating too many sugar-saturated foods typically reduces a person's consumption of healthful foods because the sugar in foods makes one feel full before having digested a sufficient amount of healthful food. Also, sugar can cause the body's natural insulin process to increase to allow the body to convert the sugar into something useful. However, continual or excessive intake of sugar dulls the insulin response such that the body works hard ineffectively. This eventually may lead to Syndrome X and Type II diabetes, when insulin is ineffective and no longer produced in a proper amount, and then a person needs artificial insulin on a daily basis.

Protein is a fundamental nutritional and dietary element that the body requires to function, grow, and heal. Protein is used for energy and for tissue development and restoration. Protein is the building block for muscles, bones, cartilage, and skin. Hair and nails, for example, are composed primarily of protein. Protein helps a person look their best physically. On the inside, protein is crucial in the creation and maintenance of every cell in the human body. Protein helps produce enzymes and other body chemicals that power the body, including aiding digestion, maintaining proper hormone levels, and distributing oxygen throughout the body.

Managing and consuming foods with the proper proportions of fiber, sugar, and protein is critical to one's health, energy levels, weight, and shape. Eating foods that are high in fiber, protein, or both is generally considered ideal.

Implementations herein provide systems and/or methods for a user to obtain or capture (e.g., via a user device such as a mobile or smart phone) nutritional information and transform the information into an easy to digest and understand format. For example, the system may obtain one or more key nutritional facts of a labeled food item. For example, the system may obtain three key nutrition facts such as quantities of fiber, sugar (total sugars and/or added sugars), and protein available in the food item (e.g., in grams). In addition, because each person has a unique physiology, medical conditions, illnesses, and/or health goals, the particular forms of sugar in a food item may be of special concern. Accordingly, the system provides for easy switching between obtaining/assessing the quantity of “added sugars” in a food item and obtaining/assessing the “total sugars” (i.e., the sum of natural sugars and sugars added during manufacture) in a food item. For example, a food item may have zero total sugars (i.e., both the natural sugars value and the added sugars value is zero), only natural sugars (i.e., the added sugars value is zero), only added sugars (i.e., the total sugars value and the added sugars value are identical), or a combination of natural and added sugars (i.e., the total sugars value is equal to a sum of the added sugars value and any natural sugars present in the food item).

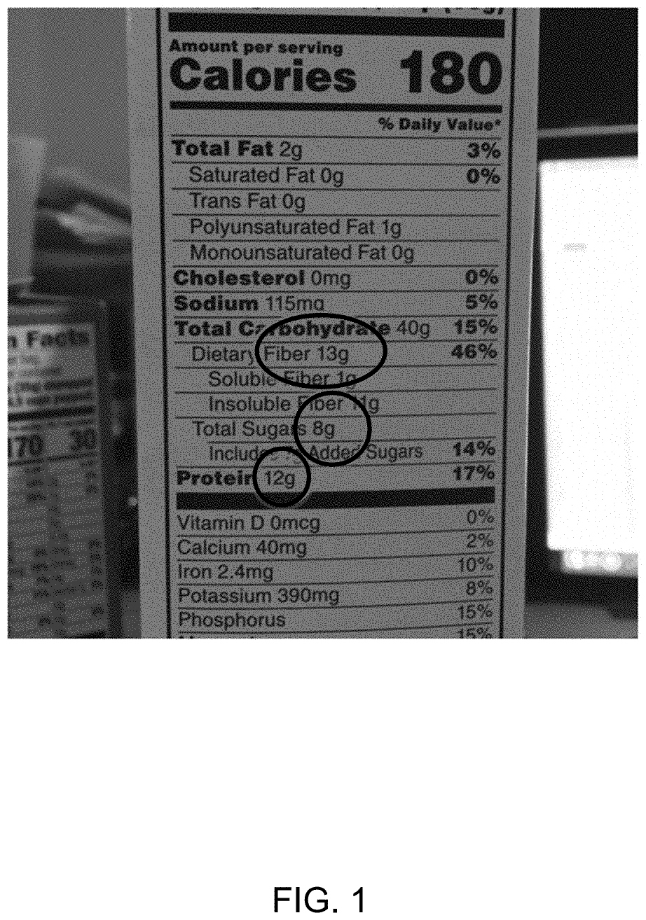

Optionally, the system may use a user device equipped with an image sensor (such as a mobile or smart phone with a camera) to capture image data representative of the food label. For example, the user may execute an application on a mobile phone and direct a field of view of the camera toward a food label (i.e., a nutrition facts table) of a food item. Optionally, the system may obtain the nutrition information by scanning the barcode of the food item. The system captures one or more images of the food label and, using imaging processing techniques, determines a plurality of nutrition values of the food item. For example, the system may employ a form of optical character recognition to recognize text relevant to the specific nutrition values. Nutrition labels typically have nutrition facts in generally the same location. For example, as shown in FIG. 1, an amount of dietary fiber, an amount of total sugars, an amount of the total sugars that are added sugars, and an amount of protein are all located in conventional locations on a standard food label or nutrition facts table. Specifically, these items are at the bottom of the primary table and are above a vitamins and minerals section. The system may use this pattern to quickly identify the desired nutrition values (e.g., the values of the dietary fiber, added sugars, total sugars, and protein).

The user may select, via the application, the desired nutrition values (i.e., the user may manually enter nutrition values via user inputs such as text fields, drop down boxes, sliders, and the like). In other examples, the nutrition values may be predetermined or preselected. The user device may capture the nutrition values in other ways. For example, the user device may include a microphone and the user may read the values aloud and the user device may capture the nutrition values via the user's voice. In other examples, the user may indicate the food item (i.e., by a brand, type, size, and/or other descriptors), and the system may retrieve the relevant nutrition values from a database (e.g., a local database or a cloud database accessed via the Internet) of stored nutritional values.

Each nutrient of the nutrition facts table (i.e., the nutrient component that corresponds with the nutrition values) is associated with a nutrition weight. The nutrition weight is indicative of a “goodness” or healthiness of the associated nutrient. Nutrients with higher weights are nutrients that are important or better in greater quantities than nutrients with lower weights. For example, fiber and protein may have a relatively high nutrition weight while sugar may have a relatively lower nutrition weight than fiber and protein, which indicates that generally a food item with greater fiber and protein than sugar is generally healthier than the inverse.

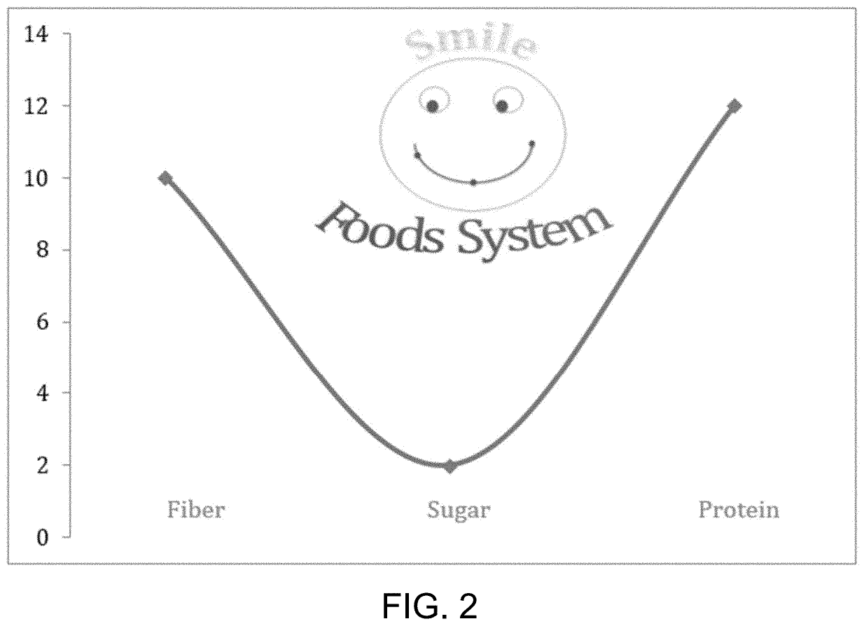

After determining the nutrition values, the system may plot or graph the nutrition values using a Cartesian graph. As shown in FIG. 2, the x-axis of the Cartesian graph may represent the different nutrients determined (e.g., fiber, sugar, protein, etc.) while the y-axis represents the amount of the nutrition component (e.g., in grams). The system orders the nutrients along the y-axis based on the corresponding nutrition weight. Optionally, the system plots nutrients with a low nutrition weight in the center of the graph and nutrients with high nutrition weight on either side of the nutrients with low nutrition weight. For example, when the three determined nutrients are fiber, sugar, and protein, the system may plot fiber first, then sugar, and then protein in that order. Fiber and protein generally have higher nutrition weights and “sandwich” sugar, which has a lower nutrition weight.

The sugar of the plot may represent the total sugar present in the food item or just the “added” sugar (i.e., the total sugar minus the natural sugar). The user may select, via the application (e.g., via a button, slider, or any other user input), whether the system uses the total sugar or added sugar. The system may allow the user to easily switch between plots/graphs using total sugar and added sugar or natural sugar (i.e., the total sugar value subtracted by the added sugar value). The system may display two separate plots simultaneously, where one plot uses total sugar while the other plot uses only added sugar or natural sugar.

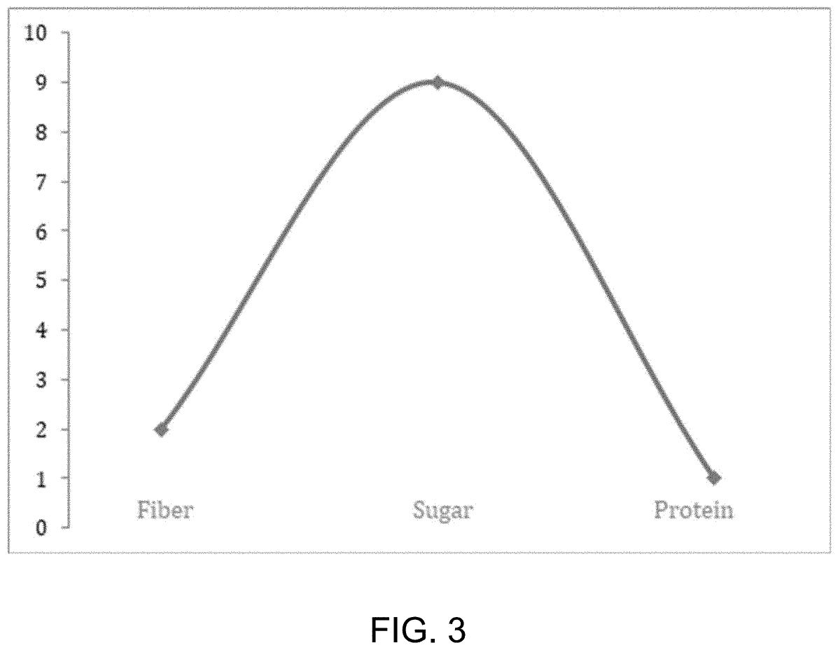

Based on the ordering of nutrients along the y-axis, the system generates a plot with easily recognizable geometric shapes or patterns that allow a user to ascertain a healthiness or quality of a food item with merely a glance. For example, as shown in FIG. 2, with the nutrient order of fiber, sugar, and protein, a healthful food item that is high in fiber and protein and low in sugar (as indicated by the food item represented by the plot of FIG. 2), when a line is fitted to the plotted points, simulates a “smile” pattern. Similarly, a food item with low fiber and low protein but high sugar plotted in this manner would simulate a “frown” shape (FIG. 3). The smile and frown shapes are easily recognizable by users as a positive or negative judgment of the quality or healthiness of a food item without the user needing to attempt to decipher the nutrition facts table. For example, the frown shape is associated with foods that are high in sugar but otherwise lack recommended nutrients, and thus should be eaten only in moderation. Thus, the system can quickly distil the nutrition facts table into one of three options. When the pattern reveals a smile, the user is informed that the food may be eaten as part of a healthful meal. When the pattern reveals a frown, the user should be wary of eating the food more than sparingly. When the pattern reveals a half-smile (i.e., a grin or partial smile, such as when the food item has substantial fiber but not substantial protein or when the food item has substantial protein but not substantial fiber), the user is informed that the food may be complementary to other foods. When the pattern reveals a partial frown (e.g., substantial fiber and sugar without substantial protein or substantial protein and sugar without substantial fiber), the user is informed that the food has some nutritious benefit but also some reason to be wary. When the pattern reveals a flat face (i.e., a straight line or non-smile/non-frown), the user is informed about the several possible ramifications of this food or drink composition.

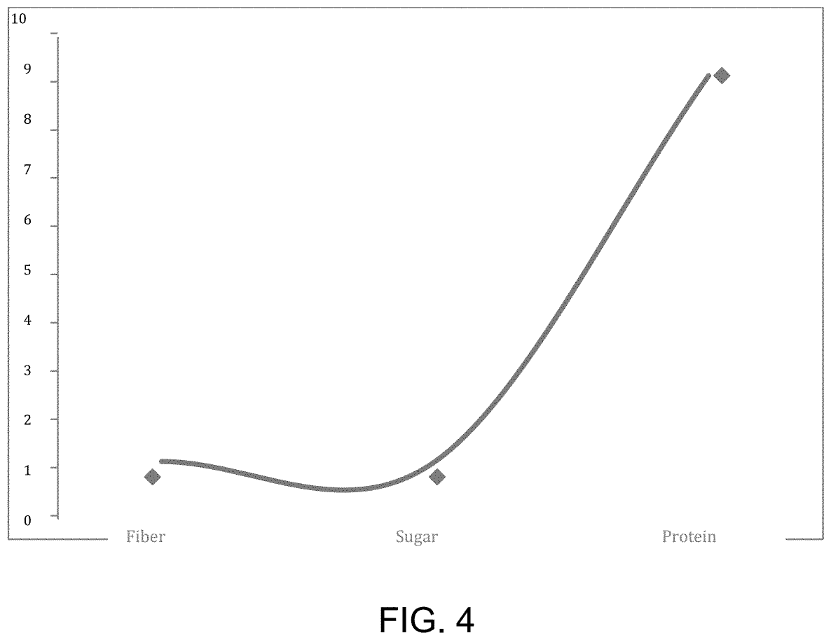

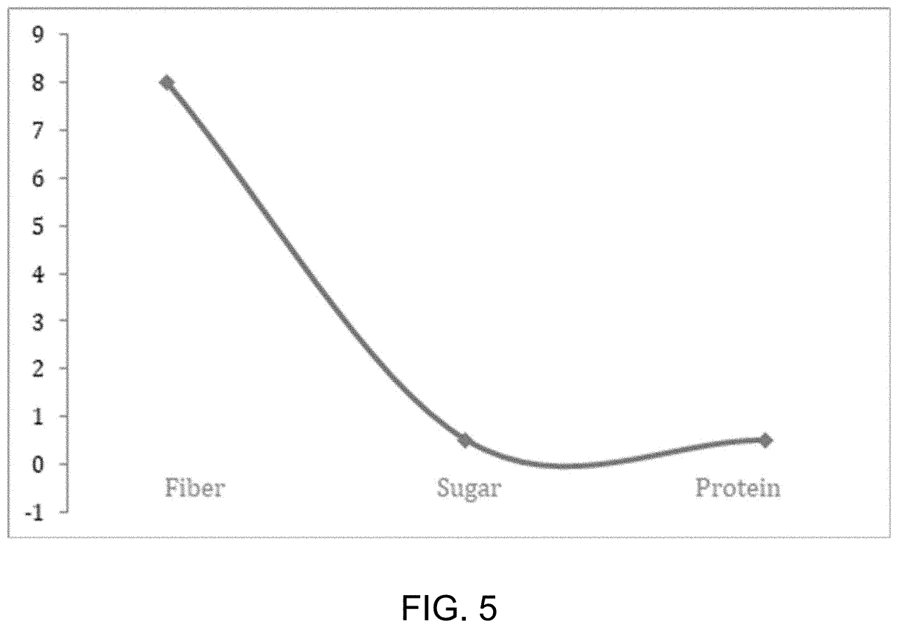

As shown in FIGS. 4 and 5, the curves fitted to the plot points of the nutritional values may form other shapes. FIG. 4 illustrates a food item that is high in protein, but low in sugar and fiber. FIG. 5 illustrates a food item that is high in fiber, but low in sugar and protein. These “half-smiles” may be combined together to form a full smile, informing a user that the two food items may be beneficial to eat together for nutritional benefit.

Examples herein illustrate the system determining three nutrition values (i.e., protein, sugar, and fiber), plotting these values on a Cartesian graph, and fitting a line to the plotted points to generate an easily recognizable shape or pattern (i.e., a smile, half-smile, or frown) to quickly and accurately distil information from a nutrition facts table to a user. However, the system may determine any number of nutrient values for any number of nutrients (e.g., fats, other carbohydrates, vitamins, minerals, etc.). The system may assign each nutrient any weight (and the user may configure each weight based on dietary needs or desires) and the system may order the nutrients in any order along the x-axis of the Cartesian graph. For example, the system may order the nutrients along the x-axis based on the weights. While a smile and frown provide an easily discernible pattern, other patterns may also be used. For example, the patterns may include a full smile, a half-smile (e.g., a grin such as a fiber grin or protein grin), a full frown, a half-frown (e.g., a partial frown such as a fiber frown or a protein frown), and a “flat face” or straight or generally straight or horizontal line (i.e., where the sugar, protein, and fiber amounts are all roughly equivalent values) The system may plot the points on the Cartesian graph in any manner (e.g., with any spacing, offsets, color, etc.).

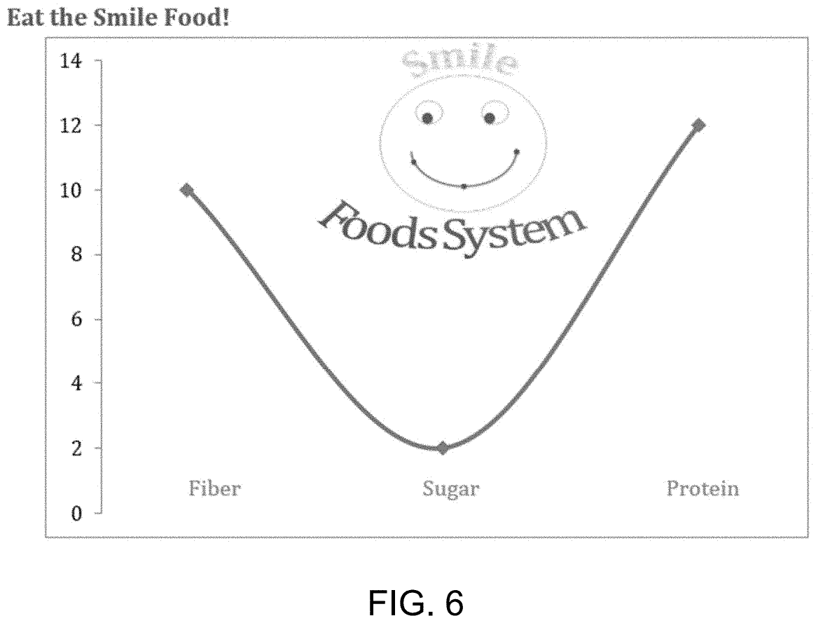

Optionally, the system includes a server or other computing device that communicates (e.g., via the Internet) with the user device (e.g., a smart phone, tablet, laptop, desktop, etc.). The server may host an application that the user device accesses (e.g., via the Internet). The application executes on the user device (e.g., in response to a user input). The application may capture a frame of image data that includes the nutrition facts table of a food item the user is interested in. The application may provide a reminder to the user to ensure the appropriate nutrient values are within the field of view of the camera. The application may briefly display the captured image (e.g., on a display of the user device) to the user. The application then extracts the nutrient values of select nutrients from the nutrient fact table displayed in the frame of image data. The system may plot these nutrient values as described above and display the plot to the user via the display of the user device. Along with the plot, the system may provide a recommendation associated with the plotted line. For example, the system may provide messages that include “eat the smile food,” “eat the half-smile food, fiber heavy,” “eat the half-smile food, protein heavy,” and/or “skip the frown food” (FIG. 6). The application may allow the user to configure nutritional weights. The application may provide the user with a number of inputs (e.g., weight, age, goals, etc.) to allow the user to smartly customize nutritional weights and/or other concerns.

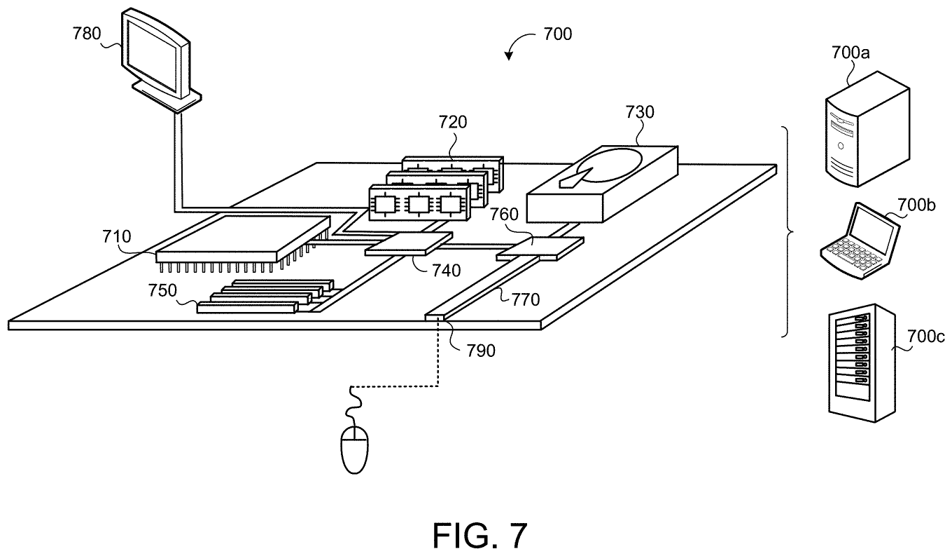

FIG. 7 is schematic view of an example computing device 700 that may be used to implement the systems and methods described in this document. The computing device 700 is intended to represent various forms of digital computers, such as smart phones, laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 700 includes a processor 710, memory 720, a storage device 730, a high-speed interface/controller 740 connecting to the memory 720 and high-speed expansion ports 750, and a low speed interface/controller 760 connecting to a low speed bus 770 and a storage device 730. Each of the components 710, 720, 730, 740, 750, and 760, are interconnected using various busses, and may be mounted on a common motherboard or in other manners as appropriate. The processor 710 can process instructions for execution within the computing device 700, including instructions stored in the memory 720 or on the storage device 730 to display graphical information for a graphical user interface (GUI) on an external input/output device, such as display 780 coupled to high speed interface 740. In other implementations, multiple processors and/or multiple buses may be used, as appropriate, along with multiple memories and types of memory. Also, multiple computing devices 700 may be connected, with each device providing portions of the necessary operations (e.g., as a server bank, a group of blade servers, or a multi-processor system).

The memory 720 stores information non-transitorily within the computing device 700. The memory 720 may be a computer-readable medium, a volatile memory unit(s), or non-volatile memory unit(s). The non-transitory memory 720 may be physical devices used to store programs (e.g., sequences of instructions) or data (e.g., program state information) on a temporary or permanent basis for use by the computing device 700. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random-access memory (RAM), dynamic-random access memory (DRAM), static random-access memory (SRAM), phase-change memory (PCM) as well as disks or tapes.

The storage device 730 is capable of providing mass storage for the computing device 700. In some implementations, the storage device 730 is a computer-readable medium. In various different implementations, the storage device 730 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 720, the storage device 730, or memory on processor 710.

The high speed controller 740 manages bandwidth-intensive operations for the computing device 700, while the low speed controller 760 manages lower bandwidth-intensive operations. Such allocation of duties is exemplary only. In some implementations, the high-speed controller 740 is coupled to the memory 720, the display 780 (e.g., through a graphics processor or accelerator), and to the high-speed expansion ports 750, which may accept various expansion cards (not shown). In some implementations, the low-speed controller 760 is coupled to the storage device 730 and a low-speed expansion port 790. The low-speed expansion port 790, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 700a or multiple times in a group of such servers 700a, as a laptop computer 700b, or as part of a rack server system 700c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, programmable logic devices [PLDs]) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Changes and modifications in the specifically described embodiments can be carried out without departing from the principles of the invention, which is intended to be limited only by the scope of the appended claims, as interpreted according to the principles of patent law including the doctrine of equivalents.

1. A method for tracking nutrition, the method comprising:
obtaining, by a user device, data representative of at least a portion of a nutrition facts table of a food item;
determining, by a processor of the user device, using the data, a plurality of nutrition values, wherein each nutrition value represents an amount of a respective nutrient present in the food item;
assigning, by the processor, a nutrition weight to each respective nutrient;
plotting, by the processor, the nutrition values on a graph based on the nutrition weights of each respective nutrient, the plot forming a pattern indicative of a healthiness level of the food item; and
displaying the plot on a display of the user device.

2. The method of claim 1, wherein the plurality of nutrition values comprises a protein nutrition value, a fiber nutrition value, and a sugar nutrition value.

3. The method of claim 2, wherein the sugar nutrition value comprises one selected from the group consisting of (i) a quantity of total sugar and (ii) a quantity of added sugar.

4. The method of claim 2, wherein the method comprises, responsive to a user input, changing the sugar nutrition value from a quantity of total sugar to a quantity of added sugar.

5. The method of claim 2, wherein plotting the nutrition values on the graph comprises plotting the nutrition values with the nutrition weight of sugar in between nutrition values of fiber and protein.

6. The method of claim 5, wherein the pattern comprises one selected from the group consisting of (i) a smile, (ii) a half-smile, (iii) a frown, (iv) a partial frown, and (v) a flat face.

7. The method of claim 1, further comprising displaying, on the plot, a message based on the pattern of the plot.

8. The method of claim 1, wherein determining the plurality of nutrition values comprises using optical character recognition.

9. The method of claim 1, wherein the graph comprises a Cartesian graph.

10. The method of claim 9, wherein the Cartesian graph comprises an x-axis of the respective nutrients and a y-axis comprises the nutrition values of the respective nutrients.

11. The method of claim 10, wherein the plurality of nutrition values comprises a protein nutrition value, a fiber nutrition value, and a sugar nutrition value, and wherein plotting the nutrition values comprises plotting the sugar nutrition value between the fiber nutrition value and the protein nutrition value.

12. The method of claim 11, wherein plotting the nutrition values comprises plotting the protein nutrition value to the right of the sugar nutrition value and plotting the fiber nutrition value to the left of the sugar nutrition value.

13. The method of claim 1, wherein obtaining the data comprises capturing, by a camera of the user device, a frame of image data representative of the at least the portion of the nutrition facts table of the food item.

14. The method of claim 13, wherein capturing the frame of image data comprises a user of the user device placing the nutrition facts table within a field of view of the camera.

15. The method of claim 1, wherein obtaining the data comprises capturing, by a camera of the user device, a frame of image data representative of the at least the portion of a bar code or QR code of the food item.

16. A method for tracking nutrition, the method comprising:
receiving, at a user device, user input;
determining, by a processor of the user device, using the user input, a plurality of nutrition values, wherein each nutrition value represents an amount of a respective nutrient present in a food item;
assigning, by the processor, a nutrition weight to each respective nutrient;
plotting, by the processor, the nutrition values on a graph based on the nutrition weights of each respective nutrient, the plot forming a pattern indicative of a healthiness level of the food item; and
displaying the plot on a display of the user device.

17. The method of claim 16, wherein the user input comprises one selected from the group consisting of (i) a voice of a user speaking to the user device and (ii) user interactions with a user interface executing on the user device.

18. The method of claim 16, wherein the plurality of nutrition values comprises a protein nutrition value, a fiber nutrition value, and a sugar nutrition value.

19. The method of claim 18, wherein the sugar nutrition value comprises one selected from the group consisting of (i) a quantity of total sugar and (ii) a quantity of added sugar.

20. The method of claim 16, wherein determining the plurality of nutrition values comprises retrieving, from a database, the plurality of nutrition values.
